(19) United States
(12) Patent Application Publication
(10) Pub. No.: US 2004/0037872 A1
(43) Pub. Date: Feb. 26, 2004

(54) Tropical composition

(76) Inventors: Isabelle Francine Liebschutz, Gevrey-Chambertin (FR); Cecile Aillaud, Talant (FR); Chantal Lapillonne, Valencin (FR)

Correspondence Address: MERCHANT & GOULD PC, P.O. BOX 2903, MINNEAPOLIS, MN, 55402-0903, US

(21) Appl. No.: 10/380,807

(22) PCT Filed: Sep. 17, 2001
(86) PCT No.: PCT/EP01/10746

(30) Foreign Application Priority Data
Sep. 18, 2000 (EP) 00120385.0

(51) Int. Cl. A61K 9/70

(52) U.S. Cl. 424/449

(57) ABSTRACT

The invention relates to an active substance-containing adhesive patch comprising diclofenac or a topically acceptable salt thereof. Said patch is composed of an outer impermeable backing layer, a matrix layer comprising the active substance and having a composition specifically adapted to ensure optimal topical administration of diclofenac, and a protective layer which can be pulled off.

The invention relates to the topical (=external) treatment of e.g. pain, inflammatory conditions and rheumatic conditions with the well-known antiinflammatory compound diclofenac and topically acceptable salts thereof.

Diclofenac is one of the most widely used drugs worldwide and is mainly beneficial to treat antinflammatory diseases including rheumatic arthritis as well as all sorts of painful conditions. Usually it Is applied perorally, e.g. in tablet or capsule form; also suppositories are available on the market. Moreover, several topical compositions comprising diclofenac or a salt thererof, like ointments, gels or emulsion-gels, are on the market for the treatment of e.g. back pain, sprains, bruises or lumbago.

Since ca.1 980 active substance-containing adhesive patches—also frequently designated as transdermal therapeutic systems (TTS's)—have been introduced on the market and become increasingly popular. For example, they included drugs like scopolamine, estradiol, nitroglycerine or nicotine. Usually, these are patches which are fixed on the skin, comprise a certain amount of drug and are capable of releasing the drug at a certain rate. The drug released penetrates through the skin to either reach blood circulation of the patient and/or the site where it Is Intended to act. The main advantage of patches over conventional topical forms like ointments or gels Is that the drug usually is released and penetrates through the skin over a much longer period of time, e.g. up to 24 h and longer. As with conventional topical compositions, using a patch can have numerous advantages depending on the kind of drug applied because passage of the drug through the gastro-intestinal tract is avoided.

Development of patches for nonsteroidal antiinflammatory drugs (NSAIDs) including diclofenac looked particularly attractive because (a) any potential problem with stomach irritation or gastric ulcer formation, which were known to be possible side effects with NSAIDs, would be avoided, and (b) it was shown before that in principle NSAIDs were capable of penetrating through the skin (cp. the topical compositions mentioned above).

It is therefore, at first sight, surprising that up to now only few and no really satisfying patches comprising diclofenac or a salt thereof are available. But when the present inventors started experimenting, it turned out rather quickly that it would be a very difficult task to obtain a satisfactory diclofenac patch. The reason was that a said diclofenac patch had to combine various properties which soon turned out to be extremely difficult to combine. Sufficient release of the active substance, diclofenac, (from the patch onto the skin surface) had to be combined with a good adhesion of the patch to the skin (over a long period of time), moreover with an as good a penetration of diclofenac through the skin as possible, and said patch had further to be non-irritating to the human skin. Another goal was that the active substance should be fully dissolved in the matrix layer of the patch, if possible. In that way, a much higher bioavailability of the active substance would be achievable, and the amount of active substance needed in the patch would be much smaller than in case that the active substance was present as a suspension in the matrix layer. Only after extensive experimentation the present inventors finally succeeded in obtaining a patch that fulfilled all requirements In a surprising and astonishing manner.

Therefore, the invention relates to an active substance-containing adhesive patch, which comprises

(a) an impermeable backing layer,

(b) a matrix layer comprising

(1) didofenac, or a topically acceptable salt thereof, in an amount of 1-15% of the total of the matrix layer,

(2) a matrix-forming polymer selected from styrene-isoprene-styrene copolymer and ethylene-vinyl acetate copolymer, in an amount of 15-42% of the total of the matrix layer,

(3) a tacidfier selected from aliphatic hydrocarbon resins and thermoplastic terpenic resins, in an amount of 42-70% of the total of the matrix layer, and

(4) one or more solv nts selected from the group consisting of oleic add and derivatives thereof, fatty acid alkyl esters and N-alkyl-pyrrolidones, in an overall amount of 2-20% of the total of the matrix layer, and

(c) a protective layer which can be pulled off.

Impermeable with respect to the backing layer (a) means that it is essentially impermeable to e.g. the active substance and water. There are many materials which are suitable for that purpose. For example, the backing layer (a) may be composed of ethylene/vinyl acetate copolymer or polyolefine foams.

The matrix layer (b) has pressure sensitive, adhesive properties and is the layer which adheres to the skin when the removable protective layer (c) is pulled off and the patch is attached to the skin of a patient.

(b)(1): The active substance for which the patch is specifically designed is diclofenac (which means the diclofenac free acid) including the topically acceptable salts thereof, e.g. the sodium salt (diclofenac sodium), the potassium salt (diclofenac potassium), the diethylammonium salt (didofenac diethylammonium) or the N-(2-hydroxyethyl)-pyrrolidinium salt (diclofenac epolamine). Preferred is diclofenac sodium.

The diclofenac component is typically present in an amount of 1-15%—preferably 1-10% and in particular 1-5%—of the total of the matrix layer. It Is a characteristic feature of the patches of the invention that the diclofenac component is usually fully dissolved in the mixture of other components forming the matrix layer. The advantage thereof Is that the bloavailability of the active substance is much higher (than in cases where the diclofenac component is suspended In the matrix layer), and consequently the overall amount of active substance In the patch can be much lower.

(b)(2): As matrix-forming polymers are used either styrene-isoprene-styrene (SIS) copolymer or ethylene-vinyl acetate (EVA) copolymer, or a combination thereof, in an amount of 15-42%—preferably 17-40% and In particular 20-40%—of the total of the matrix layer.

If SIS Is used, it preferably is present in an amount of 15-35%—more preferably 15-29%, most preferably 15-25%, especially 17-25% and in particular 20-24%—, or in an amount of 21-34%, of the total of the matrix layer.

If EVA is used, it preferably is present in an amount of 32l-42%—in particular 34-40%—of the total of the matrix layer.

(b)(3): As tackifiers are used aliphatic hydrocarbon resins and thermoplastic terpenic resins, or a combination thereof, in an amount of 42-70%—preferably 43-65% and in particular 43-62%—of the total of the matrix layer.

Aliphatic hydrocarbon resins are typically C₄-C₅(polyalkadienes, polyalkenes or polycycloalkenes) or mixtures thereof. The underlying monomers are e.g. pentadienes (1inear or branched), pentenes (linear or branched) or cyclopentene. Useful commercial products are e.g. Adtac® LV, Piocotac® 115, Piccotac® 95-E, Hercures® C, Hercures® CX, Piccopale® 100-E (all from Hercules) and Escorez® 1271 U (Exxon).

Thermoplastic terpenic resins are e.g. thermoplastic modified terpene resins, based on e.g. terpene or terpene/styrene monomers. Useful commercial products are e.g. Piocolyte®-A115, Piccolyte®-C115, Piccolyte®-S115 (all from Hercules); Sylvares® TR 7115, Sylvares® TR B125, Sylvares® ZT 5100, Sylvares® ZT 105LT and Sylvares® ZT 501 (all from Arizona Chemical).

If aliphatic hydrocarbon resins are used, it preferably Is present in an amount of 54-65% especially 54-62% and in particular 58-62%—of the total of the matrix layer.

If thermoplastic terpenic resins are used, it preferably is present in an amount of 42-50%—in particular 43-47%—of the total of the matrix layer.

The one or more solvents, (b)(4), are chosen from the group consisting of oleic acid and derivatives thereof, fatty add alkyl esters and N-alkyl-pyrrolidones, in an overall amount of typically 2-20%—preferably 6-20%, more preferably 10-20%, especially 12-18% and In particular 13-17%—of the total of the matrix layer. Typically they are liquid at room temperature.

Oleic acid, or a derivative thereof, is e.g. selected from the group consisting of oleic acid, oleic alcohol and esters of oleic acid. Esters of oleic acid are typically C₁-C₂₄-alkyl or C₂-C₂₄-alkenyl esters, e.g. ethyl oleate, decyl oleate or oleyl oleate. In particular preferred is oleic acid.

Fatty acid alkyl esters are e.g. esters of C₆-C₂₄ fatty acids with mono- or polyvalent (e.g. di- or tri-valent) alcohols, e.g. C₁-C₂₄ alkanols, ethylene glycol, propylene glycol or glycerine. In case of polyvalent alcohols, it is preferred that all hydroxy groups of the alcohol are esterified, as realized e.g. in triglycerides. Preferred are C₁-C₂₄ alkyl esters of C₆-C₂₄ fatty acids, and in particular isopropyl myristate, isopropyl stearate, isopropyl isostearate or isostearyl isostearate.

N-alkyl-pyrrolidones are typically N-C₁-C₂₄-alkyl-pyrrolidones, e.g. N-methylpyrrolidone. Preferred are N-C₆-C₂₄-alkyl-pyrrolidones, and in particular N-dodecylpyrrolidone or N octylpyrrolidone.

Preferably, the one or more solvents (b)(4) comprise oleic acid or a derivative thereof, typically in an amount of 2-10%—preferably 3-10%, especially 4-8% and in particular 5-7% —of the total of the matrix layer. More preferably, there is further present—in addition to oleic add or a derivative thereof—at least one other solvent that is selected from fatty add alkyl esters and N-alkyl-pyrrolidones. Most preferably, there is further present—in addition to oleic acid or a derivative thereof—a fatty acid alkyl ester and a N-alkyl-pyrrolidone.

The removable protective layer (c)—also called “release liner”—is pulled off prior to use of the patch. The materials which it is composed of are not critical. For example, it may be composed of siliconized polyester or PET/aluminium.

A particular embodiment of the Invention is characterized In that the active substance-containing adhesive patches as defined herein do not contain isostearic acid, specially that the matrix layers (b) thereof do not contain Isostearic acid.

A said patch can, in principle, be applied to any portion of the skin. The patches of the invention are characterised by a very good skin permeation of the drug applied. Moreover, they adhere reliably to the skin, even in case that the patient e.g. is taking a shower or is moving a joint to which the patch is attached, like the elbow. Thus, the patches of the invention are characterized by an extremely good elasticity. Further, the specific matrix composition chosen ensures that there is sufficient release of the active substance from the patch onto the skin surface for at least 24 hours. Moreover, the patches of the invention can be easily removed from the skin without leaving any residue.

The patches of the invention have valuable pharmacological properties. Especially they are beneficial in the treatment of all sorts of painful, inflammatory and rheumatic conditions, e.g. back pain, muscle pain, sprains (e.g. anide sprain), bruises, lumbago, epicondylitis, osteoarthrifis, rheumatic arthritis etc. Generally spoken, the patches of the invention are inter alia useful in all conditions for which the conventional topical diclofenac compositions on the market (like Voltaren® Emulgel® are known to be beneficial.

The beneficial properties of the patches of the Invention can be demonstrated, for example, in the following tests. Said tests may either address the beneficial galenicavitechnical properties of the patches, such as adhesion to the skin, drug penetration or drug release. For example, the in vitro drug permeation test through hairless guinea-pig skin can be mentioned here, wherein the patches of the invention show an extremely high cumulative permeation of diclofenac after 12, 24 and even 32 hours of application. Excellent results are e.g. also obtained when the In vitro drug permeation through nude mouse skin is determined. In vitro skin irritation tests e.g. on hairless guinea pig (skin tolerance guinea pig) confirm the excellent safety profile of the patches of the Invention. Moreover, measurements of the In vitro peel adhesion (substrate: a metal plate) show that the adhesion of the patches of the invention is perfectly adjusted to serve its intended purpose (of sticking reliably but being removable without problems).

On the other hand, there are many tests known in the art to demonstrate the beneficial pharmacological activity of the patches in vitro, in vivo or clinically. In that case, inter alia all the tests known to have demonstrated the beneficial properties of conventional topical diclofenac compositions on the market come Into consideration, e.g. the Carageenan-induced edema In hind paw of the rat as an assay for antiinflammatory drugs [see .g. Winter et al., Proc. Soc. Exp. Biol. Med. 111 (1962) 544-547]; the Reduction in the swelling in rats' paws in the kaolin edema test [see e.g. Helv. Physiol. Acta 25 (1967) 156 and Arzneimittelforschung 27(I) (1977) 1326]; the Inflammation induced with croton oil in the mouse ear [see e.g. Tonelli et al., Endocrinology 77 (1965) 625-634]; the Inhibition of abscess formation induced by subcutaneous injection of carageenan in rats [see e.g. Arzneimittelforschung 27(I) (1977) 1326]; or the Phenyl-p-benzoquinone writhing test (analgesia) [see e.g. J. Pharmacol. Therap. 125 (1959) 237].

The safety of the compositions of the invention is confirmed by classical toxicological studies, such as acute skin irritation on hairless guinea-pig and sensitization.

Preferably, the patches of the invention are intended for 24 hours use. Of course, it is also possible to remove them earlier, e.g. after 1, 2, 4, 8 or 16 hours. On the other hand, they may also be used longer than 24 hours, provided that the patch Is containing a sufficient amount of drug that ensures release of the drug beyond 24 hours.

The recommended duration of patch application may depend on various factors, such as the condition to be treated and the individual condition and the preferences of the patient

Moreover, the invention relates to a method of treating pain, inflammatory and rheumatic conditions, which comprises topically administering to a mammal in need of such treatment a therapeutically effective amount of diclofenac, or a topically acceptable salt thereof, in the form of a patch as defined above.

The manufacture of the topically administered pharmaceutical preparations Is effected in a manner known per se, for example by forming a solution (A) which comprises the matrix-forming polymer and the tackifier in a solvent wherein both said components are soluble, e.g. ethyl acetate, ethanol, heptane, methyl-ethyl-ketone or tetrahydrofuran. A second solution (B) Is formed where the diclofenac component Is dissolved In the solvent(s) present, optionally under the addition of an additional solvent, preferably the same one as used to form solution (A). Solutions (A) and (B) are combined and then e.g. spread evenly over the removable protective layer (“coating”), and dried, e.g. by heating In a hot air tunnel. The free side of the matrix layer (opposite to the removable protective layer) may then be laminated with the backing layer, and finally the product obtained is cut to obtain patches having the size and shape desired, and is e.g. sealed in pouches.

Another way of manufacturing the patches of the invention comprises mixing all of the components in a solvent, e.g. one of solvents mentioned above, and otherwise proceeding in an analogous manner as described above.

The following examples are intended to illustrate the invention.

### EXAMPLE 1

A patch comprising 17.5 mg of diclofenac sodium and having a size of 70 cm² has the following composition and yields the following test results.

Composition of the matrix layer

| | |
|---|---|
| (a) diclofenac sodium | 2.5% (=17.5 mg) |
| (b) styrene-isoprene-styrene copolymer | 21.0% (=147 mg) |
| (c) aliphatic hydrocarbon resin | 60.5% (=423.5 mg) |
| (d) oleic acid | 6.0% (=42 mg) |
| (e) isopropyl myristate | 10.0% (=70 mg) |

Backing layer: thin EVA foam film (600 micrometers)

Removable protective layer: siliconized polyester film (75 micrometers)

Sealable pouch: paper/aluminium/polyethylene type complex.

Experimental results:

| | |
|---|---|
| Amount of drug penetration | 3.39 ± 0.08 micrograms/cm²/h (n = 5) |
| through nude mouse skin: | |
| Accumulated amount of drug | 28.3 micrograms/cm² at 24 h (n = 2) |
| penetration through hairless | 40.9 micrograms/cm² at 32 h (n = 2) |
| guinea-pig skin: | |
| Skin irritation on hairless | well tolerated |
| guinea-pig for 4 consecutive | [AUC (“area under curve”) = 2.1] |
| days: | |
| Peel adhesion: | 338.4 N/m (n = 10). |

### EXAMPLE 2

A patch comprising 21 mg of diclofenac sodium and having a size of 70 cm² has the following composition and yields the following test results.

Composition of the matrix layer

| | |
|---|---|
| (a) diclofenac sodium | 3% (=21 mg) |
| (b) ethylene-vinyl acetate copolymer | 36% (=252 mg) |
| (c) thermoplastic modified terpenic resin | 45% (=315 mg) |
| (d) oleic acid | 6% (=42 mg) |
| (e) isostearyl isostearate | 10% (=70 mg) |

Backing layer, removable protective layer and sealable pouch: as in Example 1.

Experimental Results:

| | |
|---|---|
| Amount of drug penetration | 4.53 ± 0.55 micrograms/cm²/h (n = 5) |
| through nude mouse skin: | |
| Accumulated amount of drug | 12.4 micrograms/cm² at 24 h (n = 2) |
| penetration through hairless | 21.7 micrograms/cm² at 32 h (n = 2); |
| guinea-pig skin: | |
| Skin irritation on hairless | well tolerated (AUC = 2.0) |
| guinea-pig for 4 consecutive | |
| days: | |
| Peel adhesion: | 164.6 N/m (n = 10). |

### EXAMPLE 3

A patch comprising 21 mg of diclofenac sodium and having a size of 70 cm² has the following composition and yields the following test results.

Composition of the matrix layer

| | |
|---|---|
| (a) diclofenac sodium | 3% (=21 mg) |
| (b) styrene-isoprene-styrene copolymer | 22% (=154 mg) |
| (c) aliphatic hydrocarbon resin | 60% (=420 mg) |
| (d) oleic acid | 6% (=42 mg) |
| (e) isopropyl myristate | 5% (=35 mg) |
| (f) N-dodecylpyrrolidone | 4% (=28 mg) |

Backing layer, removable protective layer and sealable pouch: as in Example 1.

Experimental results:

| | |
|---|---|
| Amount of drug penetration | 4.66 ± 0.21 micrograms/cm²/h (n = 5) |
| through nude mouse skin: | |
| Accumulated amount of drug | 35.4 micrograms/cm² at 24 h (n = 2) |
| penetration through hairless | 52.0 micrograms/cm² at 32 h (n = 2) |
| guinea-pig skin: | |
| Skin irritation on hairless | well tolerated for 3 days; slight |
| guinea pig for 4 consecutive | reddening on day 4 on half of the |
| days: | animals (AUC = 2.3) |
| Peel adhesion: | 357.9 N/m (n = 10). |

1. An active substance-containing adhesive patch, which comprises
(a) an impermeable backing layer,
(b) a matrix layer comprising
(1) diclofenac, or a topically acceptable salt thereof, in an amount of 1-15% of the total of the matrix layer,
(2) a matrix-forming polymer selected from styrene-isoprene-styrene copolymer and ethylene-vinyl acetate copolymer, in an amount of 15-42% of the total of the matrix layer,
(3) a tackifier selected from aliphatic hydrocarbon resins and thermoplastic terpenic resins, in an amount of 42-70% of the total of the matrix layer, and
(4) one or more solvents selected from the group consisting of oleic acid and derivatives thereof, fatty acid alkyl esters and N-alkyl-pyrrolidones, in an overall amount of 2-20% of the total of the matrix layer, and
(c) a protective layer which can be pulled off;
with the proviso that said patch does not contain isostearic acid.

2. A patch according to claim 1, which is characterized by a matrix layer (b) comprising
(1) diclofenac, or a topically acceptable salt thereof, in an amount of 1-10% of the total of the matrix layer,
(2) a matrix-forming polymer selected from styrene-isoprene-styrene copolymer and ethylene-vinyl acetate copolymer, in an amount of 17-40% of the total of the matrix layer,
(3) a tackifier selected from aliphatic hydrocarbon resins and thermoplastic terpenic resins, in an amount of 43-65% of the total of the matrix layer, and
(4) one or more solvents selected from the group consisting of oleic acid, fatty acid alkyl esters and N-alkyl-pyrrolidone, in an overall amount of 12-18% of the total of the matrix layer.

3. A patch according to any one of claims 1-2, wherein the diclofenac component is selected from diclofenac free acid, diclofenac sodium, diclofenac potassium, diclofenac diethylammonium and diclofenac epolamine.

4. A patch according to any one of claims 1-3, wherein the matrix-forming polymer (b)(2) is a styrene-isoprene-styrene copolymer and is present in an amount of 15-35% of the total of the matrix layer.

5. A patch according to any one of claims 1-3, wherein the matrix-forming polymer (b)(2) is a styrene-isoprene-styrene copolymer and is present in an amount of 15-29% of the total of the matrix layer.

6. A patch according to any one of claims 1-3, wherein the matrix-forming polymer (b)(2) is an ethylene-vinyl acetate copolymer and is present in an amount of 32-42% of the total of the matrix layer.

7. A patch according to any one of claims 1-6, wherein the tackifier (b)(3) is an aliphatic hydrocarbon resin and is present in an amount of 54-65% of the total of the matrix layer.

8. A patch according to any one of claims 1-6, wherein the tackifier (b)(3) is a thermoplastic terpenic resin and is present in an amount of 42-50% of the total of the matrix layer.

9. A patch according to any one of claims 1-3, which comprises as
(b)(2) a matrix-forming polymer selected from styrene-isoprene-styrene copolymer in an amount of 15-29%, and ethylene-vinyl acetate copolymer in an amount of 32-42%, of the total of the matrix layer, and
as (b)(3) a tackifier selected from aliphatic hydrocarbon resins in an amount of 54-65%, and thermoplastic terpenic resins in an amount of 42-50%, of the total of the matrix layer.

10. A patch according to any one of claims 1-9, wherein the one or more solvents (b)(4) selected from the group consisting of oleic acid and derivatives thereof, fatty acid alkyl esters and N-alkyl-pyrrolidones, are present in an overall amount of 6-20% of the total of the matrix layer.

11. A patch according to any one of claims 1-9, wherein the one or more solvents (b)(4) comprise oleic acid or a derivative thereof, in an amount of 2-10% of the total of the matrix. layer.

12. A patch according to any one of claims 1-9, wherein the one or more solvents (b)(4) comprise oleic acid or a derivative thereof, in an amount of 3-10% of the total of the matrix layer.

13. A patch according to any one of claims 1-9, wherein the one or more solvents (b)(4) comprise oleic acid or a derivative thereof, in an amount of 3-10% of the total of the matrix layer, and at least one other solvent that is selected from fatty acid alkyl esters and N-alkylpyrrolidones.

14. A patch according to any one of claims 1-9, wherein the one or more solvents (b)(4) comprise oleic acid or a derivative thereof, in an amount of 3-10% of the total of the matrix layer, together with a fatty acid alkyl ester and a N-alkyl-pyrrolidone.

15. An active substance-containing adhesive patch, which comprises
(a) an impermeable backing layer,
(b) a matrix layer comprising
(1) diclofenac, or a topically acceptable salt thereof, in an amount of 1-15% of the total of the matrix layer,
(2) a matrix-forming polymer selected from styrene-isoprene-styrene copolymer and ethylene-vinyl acetate copolymer, in an amount of 15-42% of the total of the matrix layer,
(3) a tackifier selected from aliphatic hydrocarbon resins, in an amount of 54-65% of the total of the matrix layer, and
(4) one or more solvents selected from the group consisting of oleic acid and derivatives thereof, fatty acid alkyl esters and N-alkyl-pyrrolidones, in an overall amount of 2-20% of the total of the matrix layer, and
(c) a protective layer which can be pulled off.

16. A patch according to claim 15, wherein the matrix-forming polymer (b)(2) is a styrene-isoprene-styrene copolymer and is present in an amount of 15-29% of the total of the matrix layer.

17. A patch according to claim 15 or claim 16, wherein the one or more solvents (b)(4) comprise oleic acid or a derivative thereof, in an amount of 2-10% of the total of the matrix layer.

18. A patch according to any one of claims 11-14 and 17, wherein the term “oleic acid or a derivative thereof” means oleic acid.
